(19) United States
(12) Patent Application Publication
(10) Pub. No.: US 2010/0317985 A1
(43) Pub. Date: Dec. 16, 2010

(54) SYSTEM FOR QUANTITATIVE ASSESSMENT OF CARDIAC ELECTRICAL EVENTS

(75) Inventor: Branislav Vajdic, Monte Sereno, CA (US)

Correspondence Address: VISTA IP LAW GROUP LLP, 12930 Saratoga Avenue, Suite D-2, Saratoga, CA, 95070, US

(73) Assignee: NEWCARDIO, INC., Santa Clara, CA (US)

(21) Appl. No.: 12/484,153

(22) Filed: Jun. 12, 2009

(51) Int. Cl. A61B 5/044; A61B 5/0402

(52) U.S. Cl. 600/523; 600/509

(57) ABSTRACT

Systems and methods for characterizing aspects of an electrocardiogram signal are presented, wherein primary and secondary analysis schemas are utilized to determine the timing of the end of a signal wave, such as a descending Twave, with precision. In one embodiment, the primary analysis schema involves comparing voltage amplitudes within a given sampling window and the secondary analysis schema involves comparing the results of primary analysis for successive sampling windows. The system may comprise a processor or microcontroller embedded into a system such as an electrocardiogram hardware system, personal computer, electrophysiology system, or the like.

### FIELD OF THE INVENTION

The present invention relates to the field of medical electronics. In particular, it concerns electronic systems, devices, and methods for acquisition, processing, and presentation of diagnostic data for use with humans and animals, such as electrocardiogram data.

### BACKGROUND

Although the electrocardiogram (frequently referred to as “ECG” or “EKG”) is a universally accepted diagnostic method in cardiology, frequent mistakes are made in interpreting ECGs, because the most common approach for interpretation of ECGs is based on human memorization of waveforms, rather than using vector concepts and basic principles of electrocardiography (see Hurst, J. W., Clin. Cardiol. 2000 January; 23(1):4-13). Another problem with traditional ECG recordings is that the ECG may not provide adequate indications of electrical activity of certain regions of the heart, especially the posterior region. The timing of cardiac electrical events, and the time intervals between two or more such events, has diagnostic and clinical importance. However, medical diagnosis and drug development has been significantly limited by the lack of adequate ECG measurement tools. Furthermore, prior analysis of ECG recordings required a substantial amount of training and familiarity with reading of the recorded waveforms. There have been many attempts to extract additional information from the standard 12-lead ECG measurement when measuring the electric potential distribution on the surface of the patient's body for diagnostic purposes. These attempts have included new methods of measured signal interpretation, either with or without introducing new measurement points, in addition to the standard 12-lead ECG points.

One of the oldest approaches, vector ECG (or “VCG”) includes the improvement of a spatial aspect to the ECG (see Frank, E., An Accurate, Clinically Practical System For Spatial Vectorcardiography, Circulation 13: 737, May 1956). Like conventional ECG interpretation, VCG uses a dipole approximation of electrical heart activity. The dipole size and orientation are presented by a vector that continuously changes during the heartbeat cycle. Instead of presenting signal waveforms from the measurement points (waveforms), as it is the case with standard 12-lead ECGs, in VCG, the measurement points are positioned in such a way that three derived signals correspond to three orthogonal axes (X, Y, Z), and these signals are presented as projections of the vector hodograph onto three planes (frontal, sagittal, and horizontal). In this way, VCG represents a step towards spatial presentation of the signal, but the cardiologist's spatial imagination skills were still necessary to interpret the ECO signals, particularly the connection to the heart anatomy. Furthermore, a time-dependence aspect (i.e., the signal waveform) is lost with this procedure, and this aspect is very important for ECG interpretation. VCG introduces useful elements which cannot be found within the standard 12-lead ECG, however, the incomplete spatial presentation and loss of the time dependence are major reasons why VCG, unlike ECG, has never been widely adopted, despite the fact that (in comparison to ECG) VCG can more often correctly diagnose cardiac problems, such as myocardial infarction.

There have been numerous attempts to overcome the drawbacks of the VCG method described above. These methods exploit the same signals as VCG (X, Y, Z), but their signal presentation is different than the VCG projection of the vector hodograph onto three planes. “Polarcardiogram” uses Aitoff cartographic projections for the presentation of the three-dimensional vector hodographs (see Sada, T., et al., J. Electrocardiol. 1982; 15(3):259-64). “Spherocardiogram” adds information on the vector amplitude to the Aitoff projections, by drawing circles of variable radius (see Niederberger, M., et al., J. Electrocardiol. 1977; 10(4):341-6). “3D VCG” projects the hodograph onto one plane (see Morikawa, J., et al., Angiology, 1987; 38(6):449-56. “Four-dimensional ECG” is similar to “3D VCG,” but differs in that every heartbeat cycle is presented as a separate loop, where the time variable is superimposed on one of the spatial variables (see Morikawa, J., et al., Angiology, 1996; 47: 1101-6.). “Chronotopocardiogram” displays a series of heart-activity time maps projected onto a sphere (see Titomir, L. I., et al., Int J Biomed Comput 1987; 20(4):275-82). None of these modifications of VCG have been widely accepted in diagnostics, although they have some improvements over VCG.

Electrocardiographic mapping is based on measuring signals from a number of measurement points on the patient's body. Signals are presented as maps of equipotential lines on the patient's torso (see McMechan, S. R., et al., J. Electrocardiol. 1995; 28 Suppl:184-90). This method provides significant information on the spatial dependence of electrocardiographic signals. The drawback of this method, however, is a prolonged measurement procedure in comparison to ECG, and a loose connection between the body potential map and heart anatomy.

Inverse epicardiac mapping includes different methods, all of which use the same signals for input data as those used in ECG mapping; and they are all based on numerically solving the so-called inverse problem of electrocardiography (see A. van Oosterom, Biomedizinisch Technik., vol. 42-El, pp. 33-36, 1997). As a result, distributions of the electric potentials on the heart are obtained. These methods have not resulted in useful clinical devices.

Cardiac electrical activity can be detected at the body surface using an electrocardiograph, the most common manifestation of which is the standard 12-lead ECG. Typical ECG signals are shown in present FIG. 1. The P-wave (2) represents atrial depolarization and marks the beginning of what is referred to as the “P-R interval”. The QRS complex (4) represents depolarization of the ventricles, beginning with QRS onset after the PR segment (5) and ending at a point known as the “J point” (6). Ventricular repolarization begins during the QRS and extends through the end of the Twave (14), at a point which may be termed “Tend” (8). The S-T segment (10) extends from the J point (6) to onset or start of the Twave (12). The Twave (14) extends from the Twave onset (12) through Tend (8). U waves (not shown) are present on some ECGs. When present, they merge with the end of the Twave or immediately follow it.

Physiologically, the Twave is the ECG manifestation of repolarization gradients, that is, disparities in degree of repolarization at a particular time point between different regions of the heart. It is likely that the Twave originates primarily from transmural repolarization gradient (see Yan and Antzelevitch; Circulation 1998; 98:1928-1936; Antzelevitch, J. Cardiovasc Electrophysiol 2003; 14:1259-1272.) Apico-basal and anteriorposterior repolarization gradients may also contribute (see Cohen I S, Giles W R, and Noble D; Nature. 1976; 262:657-661).

Transmural repolarization gradients arise because the heart's outer layer (epicardium) repolarizes quickly, the mid-myocardium repolarizes slowly, and the inner layer (endocardium) repolarizes in intermediate fashion. Referring again to FIG. 1, during the S-T segment (10), all layers have partially repolarized to a more or less equal extent, and the ST segment (10) is approximately isoelectric. A Twave (14) begins at a position which may be termed “Ton” (12), when the epicardial layer moves toward resting potential ahead of the other two layers. At the peak of the Twave (Tpeak) (16), epicardial repolarization is complete and the transmural repolarization gradient is at its maximum. Subsequently, endocardial cells begin their movement towards resting potential, thereby narrowing the transmural gradient and initiating the downslope of the Twave.

Finally, the M cells repolarize, accounting for the latter part of the Twave downslope. The Twave is complete at Tend (8) when all layers are at resting potential and the transmural gradient is abolished.

The QT interval (9) may be estimated from an ECG by measuring time from the end of the PR segment (5) to Tend (8). Abnormalities in the QT interval often mark susceptibility to life-threatening arrhythmias. Such abnormalities may be associated with genetic abnormalities, various acquired cardiac abnormalities, electrolyte abnormalities, and certain prescription and nonprescription drugs. An increasing number of drugs have been shown to prolong the QT interval and have been implicated as causes of arrhythmia. As a result, drug regulatory agencies are conducting increasingly detailed review of drug-induced abnormalities in cardiac electrical activity. The accuracy and precision of individual measurements is highly important for clinical diagnosis of heart disease and for evaluation of drug safety. Drug regulatory bodies worldwide now require detailed information regarding drug effects on cardiac intervals measured from ECG data (see M. Malik, PACE 2004; 27:1659-1669; Guidance for Industry: E14 Clinical Evaluation of QT/QTc Interval Prolongation and Proarrhythmic Potential for Non-Antiarrhythmic Drugs, http://www.fda.gov/cder/guidance/6922fnl.pdf).

Improved measurement accuracy and precision would reduce the risk of clinical error and the amount of resources required during drug development to meet regulatory requirements. This is particularly true for QT interval measurement. Problems in manual QT interval determination result in part from lead selection. Measured QT intervals can vary significantly depending upon the ECG lead selected for measurement. Another common problem is finding Tend. This is usually defined as the point at which the measured voltage returns to the isoelectric baseline. However, Twaves are often low-amplitude, morphologically abnormal, fused with a following U-wave, or obscured by noise. The same may apply to J-points, P-waves, U-waves and other important cardiac events.

Thus, accurate and reproducible procedures for cardiac interval measurement are urgently needed. The subject invention addresses this challenge with a relatively noise-tolerant solution for determining the timing of cardiac electrical events.

### SUMMARY

One embodiment of the invention is directed to a system for determining a signal wave transition point, the system comprising a memory device configured to store data pertinent to one or more signal waves sampled from electrodes operably coupled to one or more tissue structures; and a processor operably coupled to the memory device and configured to access the data and determine a transition point associated with the one or more signal waves, the processor configured to sample a first plurality of points of a signal wave in a first time window, the first plurality comprising at least a first-in-time point and a last-in-time point within the first time window; sample a second plurality of points of the signal wave in a second time window different in time from the first time window, the second plurality comprising at least a first-in-time point and a last-in-time point within the second time window; compare the values of the first plurality relative to each other to determine whether an intra-window patterning rule has been broken; and conduct a secondary analysis subsequent to determining that an intra-window patterning rule has been broken, the secondary analysis comprising comparing the values of the second plurality relative to each other to determine whether the intra-window patterning rule has been broken.

The system may further comprise a display operatively coupled to the processor, wherein the processor is further configured to cause a graphical image of at least one of the one or more signal waves to be depicted on the display, including a graphical indication of a respective pertinent transition point on each of the one or more signal waves. The processor and memory device of the system may be operatively coupled to one or more printed circuit boards, one of which may comprise a card having a common housing and an electronic interface bus, or be configured to interface with a personal computer. The processor and memory device may comprise an application specific integrated circuit configured to be embedded into a parent electronic device. In one embodiment, one of the processor and memory device may comprise a field programmable gate array configured to be embedded into a parent electronic device. The processor and memory device may be operatively coupled to an operating room electronic device such as an electrophysiology mapping system, an echocardiography system, or a fluoroscopic imaging system. Such operative coupling may be by an Ethernet connection, and various devices may be configured to communicate with each other via a protocol selected from the group consisting of TCPIP, FTP, and HTTP. The processor and memory device comprise a personal computer, and/or may be operatively coupled to an analog signal acquisition system, which may be operatively coupled to one or more electrodes. Such analog signal acquisition system may be, for example, an electrocardiogram system, an electroencephalogram system, or an electromyogram system. In one embodiment the processor and memory device are enclosed within an implantable housing. The memory device may be operatively coupled to an external computing system and configured to exchange data with the external computing system by wire, or wirelessly. In one embodiment the analog signal acquisition system comprises an ambulatory Holter monitor.

The processor may be configured to sample the second plurality of points in the second time window at least partially forward in time from the first time window. The signal wave containing the second plurality of points being sampled may be descending in amplitude versus time, and a signal wave transition point may be determined by the processor based upon an end of a descent of the descending signal wave. The signal wave containing the second plurality of points being sampled may be ascending in amplitude versus time, and a signal wave transition point may be determined by the processor based upon an end of an ascent of the ascending signal wave.

In another embodiment, the processor may be configured to sample the second plurality of points in the second time window at least partially backward in time from the first time window. The signal wave containing the second plurality of points being sampled may be descending in amplitude versus reverse time, and a signal wave transition point may be determined by the processor based upon an end of a descent of the descending signal wave in reverse time. The signal wave containing the second plurality of points being sampled may be ascending in amplitude versus reverse time, and a signal wave transition point my be determined by the processor based upon an end of an ascent of the ascending signal wave in reverse time.

### BRIEF DESCRIPTION OF THE DRAWINGS

FIG. 1 illustrates aspects of a conventional ECG signal.

FIGS. 2A-2D illustrate window-based sampling and analysis of certain aspects of a descending Twave of an ECG signal.

FIG. 3 illustrates various aspects of an ECG signal analysis configuration whereby a QT interval may be measured in accordance with the present invention.

FIG. 4 illustrates various aspects of an ECG signal analysis configuration whereby a QT interval may be measured in accordance with the present invention.

FIG. 5 illustrates various aspects of an ECG signal analysis configuration whereby a QT interval may be measured in accordance with the present invention.

FIG. 6 illustrates various aspects of an ECG signal analysis configuration whereby affects of medication may be analyzed and utilized in adjusting treatment in accordance with the present invention.

FIG. 7 illustrates various aspects of an ECG signal analysis configuration whereby a signal wave fiducial position may be measured with reverse time analysis in accordance with the present invention.

FIG. 8 illustrates various aspects of a generalized signal wave analysis configuration whereby a signal wave fiducial position may be measured for a signal wave that is ascending in amplitude, descending in amplitude, or flat and changing in amplitude, in forward and/or reverse time, in accordance with the present invention.

FIG. 9 depicts an ECG system which may be integrated with aspects of the present invention.

FIG. 10 depicts an ambulatory Holter monitor system which may be integrated with aspects of the present invention.

FIG. 11 depicts an electrophysiology mapping system which may be integrated with aspects of the present invention.

FIG. 12 depicts an echocardiography system which may be integrated with aspects of the present invention.

FIGS. 13A and 13B depict fluoroscopy-based systems which may be integrated with aspects of the present invention.

### DETAILED DESCRIPTION

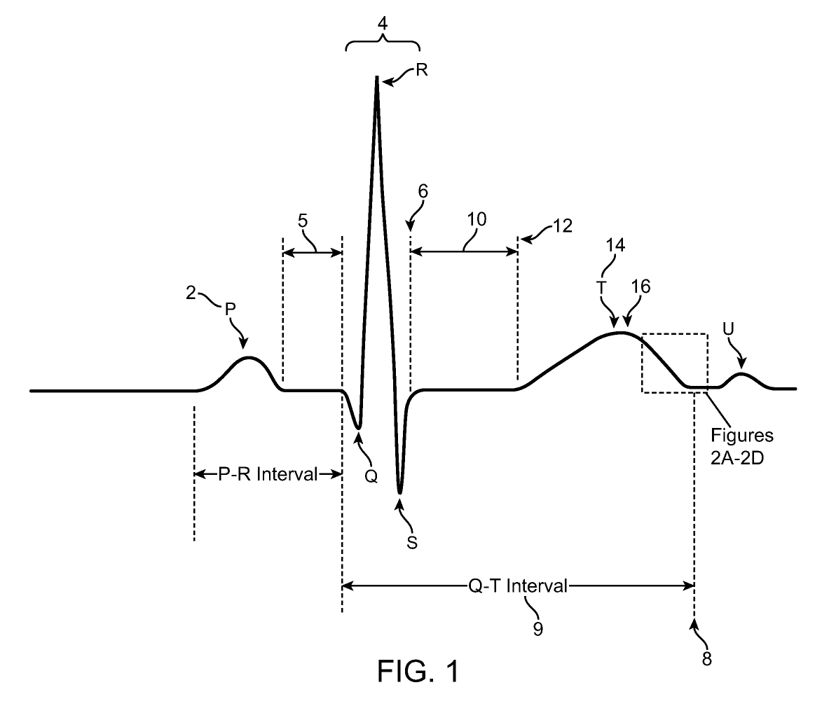

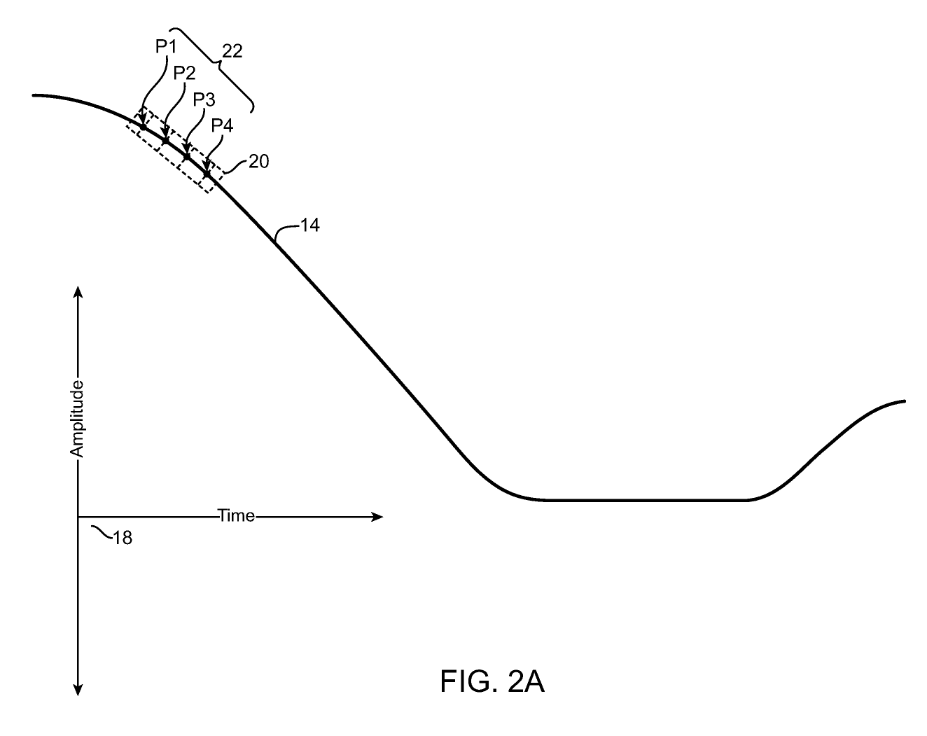

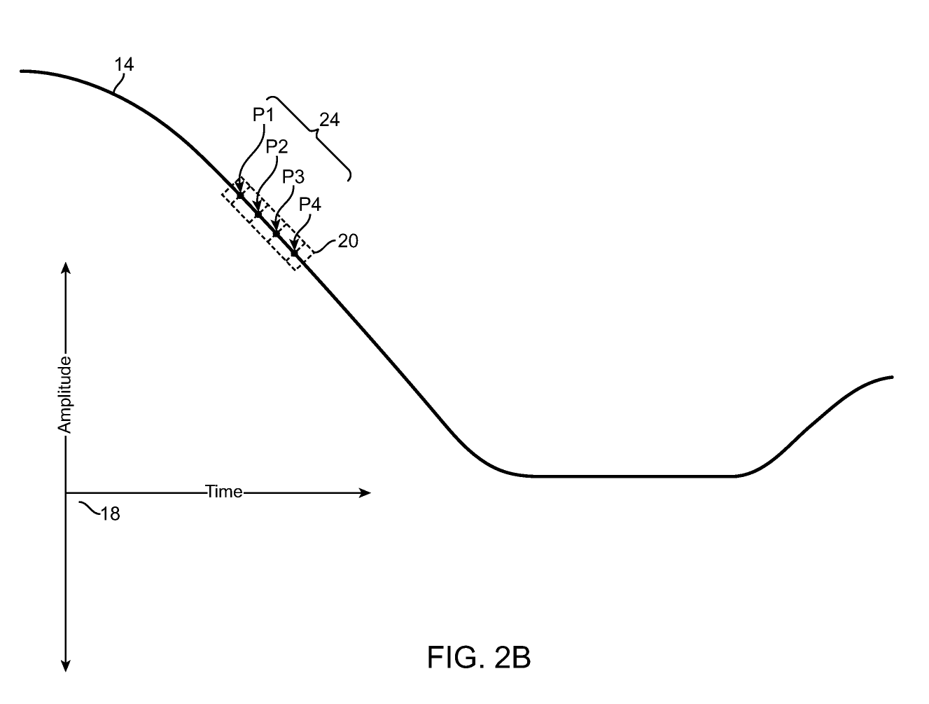

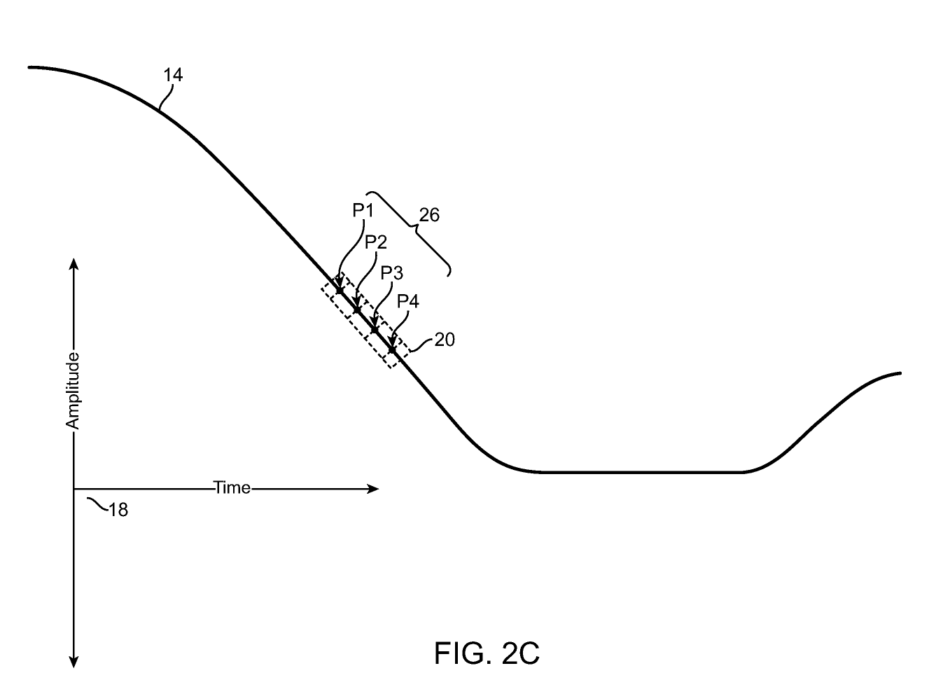

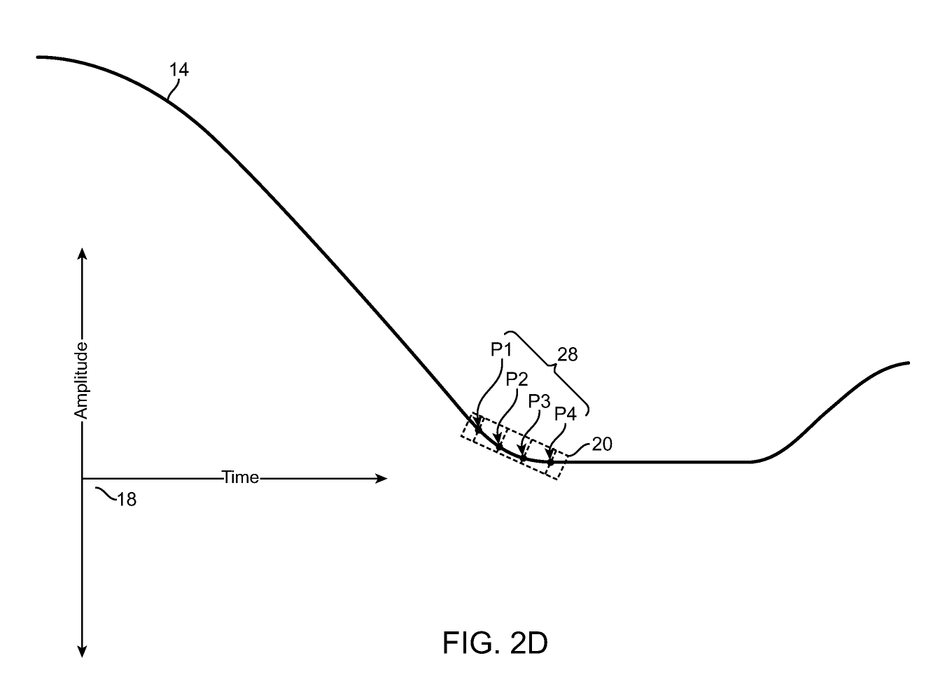

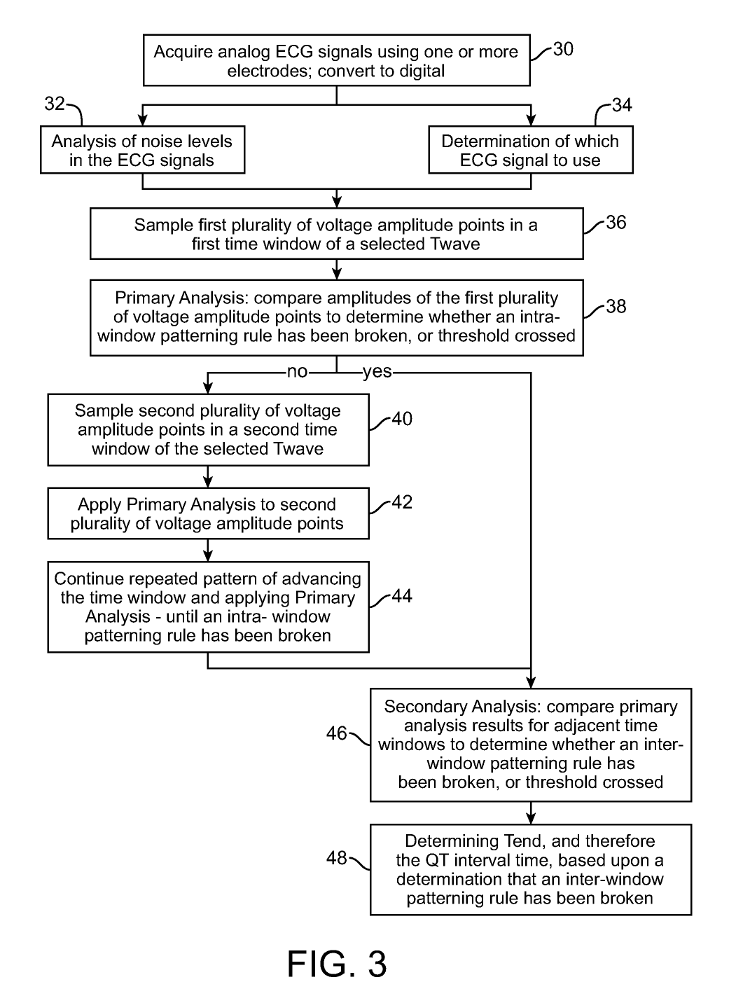

Referring to FIGS. 2A-2D, close of up views of a Twave (14), such as that shown in FIG. 1, are depicted to illustrate how Tend may be determined utilizing the data pertinent to a descending Twave signal in accordance with the present invention. Referring to FIG. 2A, a portion of a falling Twave is depicted, adjacent to a voltage amplitude versus time coordinate system axis (18). If the depicted Twave is indeed falling, one would assume that points sampled more forward in time would have smaller amplitude—and this is true for the depicted plurality of four sampled points (22) in the depicted “sampling window” (20). Indeed, referring to FIG. 2B, as the sampling window (20) is advanced forward in time to capture a different plurality of points (24), the trend continues, and more forward-in-time amplitudes are smaller. Referring to FIG. 2C, the sampling window (20) has been advanced forward in time again relative to the coordinate axes (18), and again the more forward points in this captured plurality of points (26) are smaller in amplitude. Referring now to FIG. 2D, an apparent inflection point has been reached in the descending Twave (14) as the sampling window (20) has been advanced forward yet again to capture a fourth plurality of points (28), and it is apparent from the Twave amplitudes within the sampling window (20) that at least two of the four sampled points in this sampled plurality (28) have amplitudes that are nearly equivalent. In contrast to tangent based techniques, or those which rely upon finding an intersection with a baseline, in one embodiment of the present invention, comparisons between amplitudes sampled within a given sampling window lead not to an immediate determination of a Tend—but rather to a second level of analysis. A multi-tiered approach using window-based sampling analysis with descending signals to find an endpoint such as Tend is illustrated in FIG. 3.

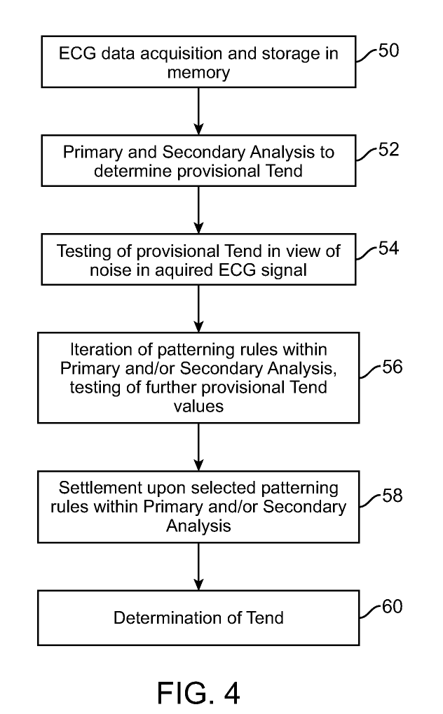

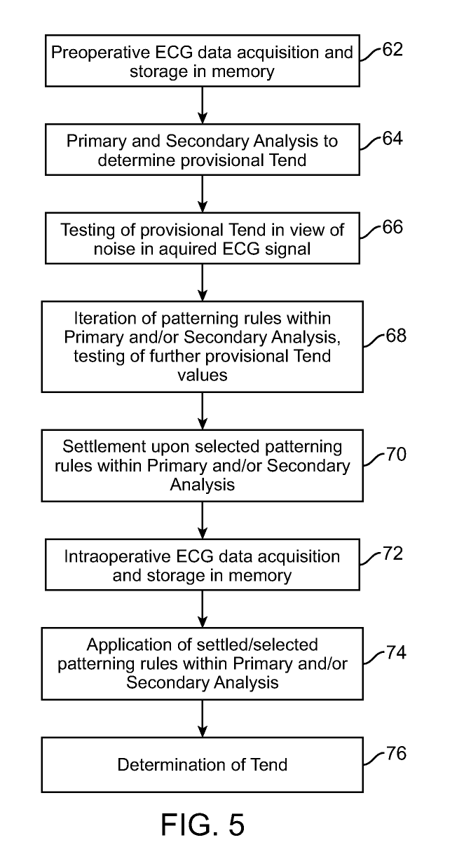

Referring to FIG. 3, raw ECG data is sampled (30) with one or more electrodes, such as a conventional set of twelve, and converted using a digital to analog converter to result in a stream, array, or set of points pertinent to each electrode, preferably in units of voltage amplitude versus time, as with the signals illustrated in FIGS. 2A-2D. Preferably the sampling is done at a high frequency, such as 250 to 500 Hz. Such high frequency sampling provides not only a high fidelity sampling of the voltage amplitude data associated with the pertinent electrode—but also provides a high fidelity representation of noise associated with such signal as well. As shown in FIG. 3, simultaneous or sequential analysis of the noise levels (32) and determination of which ECG signal to use for Tend analysis (34) precede window based analysis of the selected Twave (36). Noise analysis and signal selection are discussed further below in reference to FIGS. 4 and 5. Referring again to FIG. 3, in a manner similar to that illustrated in reference to FIG. 2A, a first plurality of voltage amplitude points may be sampled (36) using a first time window position on a selected Twave. While the graphical windowing and sampling shown, for example, in FIGS. 2A-2D is helpful for illustrative purposes, in a preferred embodiment, such analysis is conducted numerically utilizing a computer and the raw sampled data (30). Having sampled the first plurality of points (36), a primary analysis may be conducted to compare the amplitudes of the points comprising the sampled plurality. In one embodiment, the amplitude of the first-in-time (first-in-time being defined as the rightmost point using the amplitude vs. time axis (18) depicted in FIGS. 2A-2D) point comprising this plurality, such as the point labeled “P4” in the plurality (22) illustrated in FIG. 2A, may be compared with any or all of the adjacent points comprising the plurality, or more simply the last-in-time (last-in-time being defined as the leftmost point using the amplitude vs. time axis (18) depicted in FIGS. 2A-2D) point comprising this plurality, such as the point labeled “P1” in the plurality (22) illustrated in FIG. 2A. Such comparison preferably involves an “intra-window” (i.e., pertinent to points within the subject window) patterning rule, or “threshold”, and results in a determination as to whether such rule has been broken, or threshold exceeded. For example, in one embodiment, a primary analysis intra-window patterning rule dictates that the most forward in time (i.e., rightmost on the time axis (18)) point should have an amplitude less than that of the most back in time (i.e., leftmost on the time axis (18)) point within a plurality. The amplitude of P4 may simply be subtracted from that of P1, and a determination made of whether the resultant value is positive or not. In another embodiment, the resultant value need not be negative or zero to represent a broken rule or crossed threshold. For example, in one embodiment, the system may be configured to find a broken rule or crossed threshold in the event that P4 is within 10%, 20%, 30%, or other fractions of the value of P1.

In another embodiment, a primary analysis intra-window patterning rule dictates that the average amplitude of the two most forward in time points within a plurality must be less than the average amplitude of the two most back in time points within the plurality. The sampling window (20) may be configured to capture a small number of points, such as two, or a larger number of points, such as 4, 5, 6, or more.

In another embodiment, a primary analysis intra-window patterning rule dictates that a polynomial curve fit through the plurality of points should have a slope between the two most forward in time points of the plurality should have a slope not more than a certain percentage more positive than the slope of the curve between the two most back in time points of the plurality.

Referring again to FIG. 3, if the primary analysis rule has not been broken or threshold crossed, the imaginary sampling window (20) is advanced forward in time, as in the difference between FIGS. 2B and 2A, and a second plurality of points is captured (40), as described, for example, in reference to FIG. 2B. Primary analysis (42) is conducted on the second plurality of points, preferably using the same intra-window patterning analysis as above (38). This process of continuing to advance the sampling window and conducting primary analysis may be repeated (44) until a rule is broken or threshold is crossed, after which secondary analysis preferably is conducted (46), whereby primary analysis results for adjacent time windows are compared—in view of an “inter-window” (i.e., one window versus another window) patterning schema, to determine whether such rule has been broken or threshold exceeded. The inter-window rule or threshold preferably is configured to compare results of intra-window analysis for adjacent windows. For example, in one embodiment where computing resources are readily available, primary analysis may be conducted on the entire descending Twave dataset. Then, observing the data from the left to the right on the time/amplitude axis (18 in FIGS. 2A-2D), the first intra-window patterning rule breakage results in inter-window secondary analysis with regard to all of the sampling windows and pertinent pluralities of points adjacent to, or immediately adjacent to, the sampling window that caused the first intra-window patterning rule breakage. In one embodiment, more than two immediately consecutive intra-window rule breakages is associated with an inter-window rule breakage. In another embodiment, more than three primary analysis rule breakages out of a immediately consecutive grouping of five timing windows is associated with an inter-window rule breakage. In other words, the tolerance for noise and secondary analysis rule breakage may be customized—such as 2 in a row, 3 out of 5, 5 out of 7, 3 in a row, etc. Referring again to FIG. 3, Tend, and therefore the QT interval time given the QT starting point, may be determined as the time at which the inter-window rule was broken in the secondary analysis.

Referring now to FIG. 4, an embodiment is depicted wherein iteration is utilized to refine the determination of Tend. As shown in FIG. 4, raw ECG data preferably is acquired and stored in memory (50) using a device or system such as those available from GE Medical Systems under the tradename Prucka®. Primary and secondary analysis may then be utilized to determine a provisional Tend (52) based upon rule breakages pertinent to applicable intra-window and inter-window rules and data analysis. Given a provisional Tend, additional analysis may then be conducted to test its viability in view of factors such as noise in the raw data (54). For example, in one embodiment, a polynomial curve may be fitted through the raw data, and root mean square (“RMS”) analysis may be conducted to quantitatively characterize the error in view of the curve. If the position of the provision Tend is too far from that dictated by a simple baseline, tangent, or other analysis conducted on the fitted curve, and/or if the data is particularly noisy in view of the RMS analysis, the primary and secondary analysis patterning rules may be adjusted, with further determination of a second provisional Tend (56). For example, in the event that the raw data for a particular Twave is significantly noisy, a very conservative primary analysis intra-window rule may trigger secondary analysis too soon. Further, even if an appropriately tuned primary analysis intra-window rule is utilized or iterated to, thereby resulting in secondary analysis at desirable time, if the secondary analysis inter-window rule is too conservative or not conservative enough, it may cause too early, or too late, a determination of Tend. Preferably a computing system is configured to conduct such iteractive analysis, and with the input of predetermined logic based upon experience and empirical data, such processing preferably is automated, resulting in settlement upon selected patterning rules for primary and/or secondary analysis (58), and ultimately a determination of Tend.

As described above in reference to FIG. 3, one of the challenges with raw ECG signal is, indeed, noise, and twelve electrodes of potentially noisy data in high fidelity from high-frequency sampling present a challenge in determining an accurate Tend for the subject patient. In one embodiment, noise may be filtered out using statistical techniques based upon a relatively large sample size, for example as a result of a 24 hour ECG monitoring study with ambulatory hardware such as a Holter type monitor. For each electrode, a relatively large sample size of signal patterns will be available for analysis, and statistical outliers may be removed from the primary dataset and marked for further analysis, for example by cardiologists interested in drug-related Twave morphology changes. Typically, however, absent a suspected arrhythmia, a very large sample size may not be available. In other embodiments, known filtering and smoothing techniques may also be employed.

In further embodiments, vector magnitude signals, as described above, may be desirable, due to the fact that they inherently cancel out a lot more noise than raw ECG lead data. One of the challenges with vector magnitude based analysis, however, is its reliance upon accurate data for the zero reference marker at the outset of the QT interval. The end of the P-R segment (element 5 in FIG. 1) typically is marked more cleanly in an ECG signal than is Tend, but if the associated electrodes are not well attached, the signal may at least in part be coming from muscle tremor noise or environmental noise, and determining the end of the P-R segment may be more challenging. In one vector magnitude based embodiment, a compromise is made between fairly noisy analysis based upon a relatively large number of electrode signals, and reference point reliance with vector magnitude signals: primary and secondary analysis are executed for each of the X, Y, and Z vector component Twaves—because their shapes are not dependent upon the reference point (the reference point may shift such Twaves up or down in amplitude, but will not change their shape). The result is three relatively accurate measurements for Tend for each of the X, Y, and Z vector component Twaves. In one embodiment, the QT interval is determined using the furthest out Tend from the secondary analyses of the X, Y, and Z vector component Twaves. In another embodiment, the pertinent X,Y,Z coordinate system may be rotated to produce simultaneous Tend times for each of the X, Y, and Z vector component Twaves (i.e., rotate the coordinate system to a position wherein the minimum difference between the three is achieved), resulting in a single Tend determination.

Referring to FIG. 5, another embodiment is depicted to illustrate that, in practice, various aspects of the analysis may be conducted at various times relative to patient care. Referring to FIG. 5, an ECG signal preferably is acquired and stored into memory in a preoperative environment (62) without all of the same variables present intraoperatively, such as anesthesia, antithrombogenic medicines, etc. Primary and secondary analysis to determine a provisional Tend (64), along with testing of the provisional Tend in view of noise and other factors (66), as described above in reference to FIG. 4, may be conducted instantaneously or subsequent to the acquisition, depending upon computing resources, patient care timing, the need to iterate or gather more data from a particularly noisy-appearing ECG signal, etc. Similarly, iteration of patterning rules for primary and secondary analysis (68) and settlement upon a selected patterning paradigm (70) may be conducted subsequent to data acquisition, or with the patient remaining available for further data acquisition. Such settled paradigm may then be preserved and utilized (74) in subsequent scenarios with the particular patient, such as additional outpatient visits or surgical intervention, to provide expedient and refined determination of Tend (76).

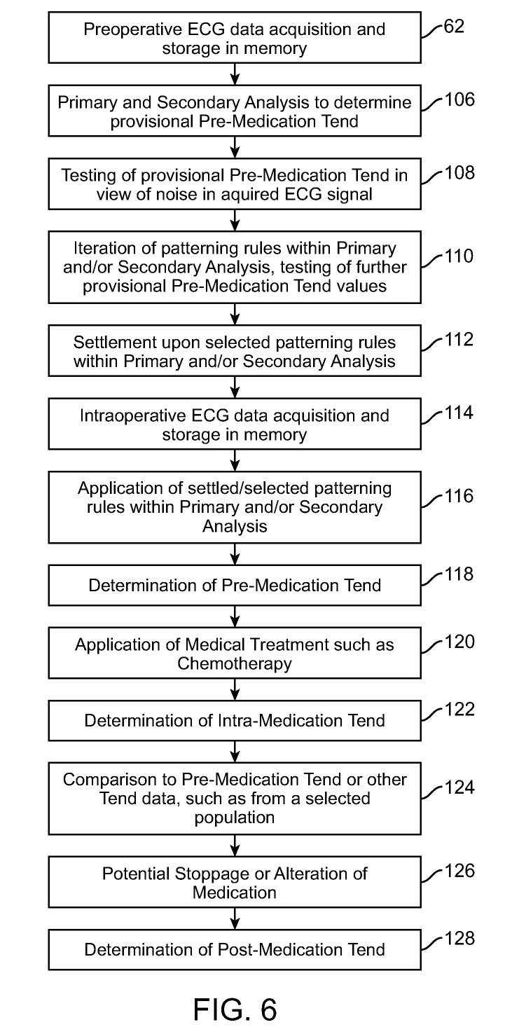

Referring to FIG. 6, an embodiment is illustrated wherein signal processing paradigms such as those described above in reference to FIGS. 2A-5 may be utilized to assist in the administration and interpretation of medical treatment. As shown in FIG. 6, subsequent to preoperative ECG data acquisition and storage in memory (62), primary and secondary analysis may be conducted to determine provisional pre-medication (i.e., before the administration of a particular medicine or medical treatment) values for ECG timing fiducials such as the end of a particular patient's Twave, Tend (106). As described above and in reference to FIG. 5, provisional values for fiducials such as Tend may be tested in view of noise levels in the acquired ECG signal wave (108), and iteration may be conducted to improve upon the patterning rules within the primary and secondary analysis with testing of further evolved provisional fiducial timing values (110), followed by settlement upon selected patterning rules within the primary and secondary analysis (112), and settlement upon a preoperative fiducial timing value which may be used for later comparison. Subsequent to this analysis, in an intraoperative or clinical environment wherein the patient is operatively coupled to the subject system for analysis, further ECG data is acquired and stored into memory (114). The settled primary and secondary patterning rules may then be applied to conduct primary and secondary analysis upon newly acquired data (116), and a pre-medication fiducial timing value, such as the timing position of Tend, determined (118); such value may be used as a “control” value for later comparison. In another variation, the settlement fiducial timing value determined using the preoperative data may also be utilized as a “control” value for comparison purposes. After application of medical treatment, such as the ingestion, injection, or other delivery of one or more chemotherapy or other medicines to the subject patient (120), the selected primary and secondary patterning rules may be applied in primary and secondary analysis to determine an intra-medication (i.e., during the medical treatment, or after administration of a particular round of medication) fiducial position, such as the timing value for Tend (122). The intra-medication values may be compared not only to the preoperative, and intraoperative-but-pre-medicinal values for the same patient, but also to those of a population, such as a population of data values from a selected healthy human population (124), and the resultant comparative information may be utilized by the medical staff to potentially alter, stop, or otherwise affect the medical treatment going forward (126), after which further ECG data may be acquired to monitor downstream conditions and watch for any post-medication (i.e., downstream in time from the previous round of medicinal intervention) changes (128).

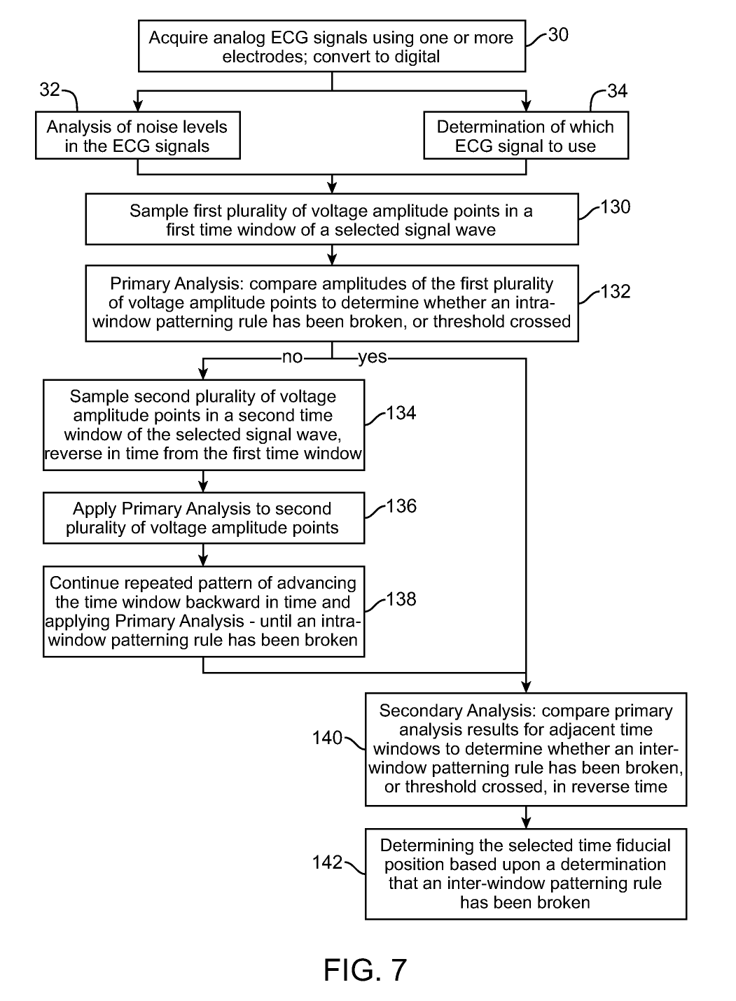

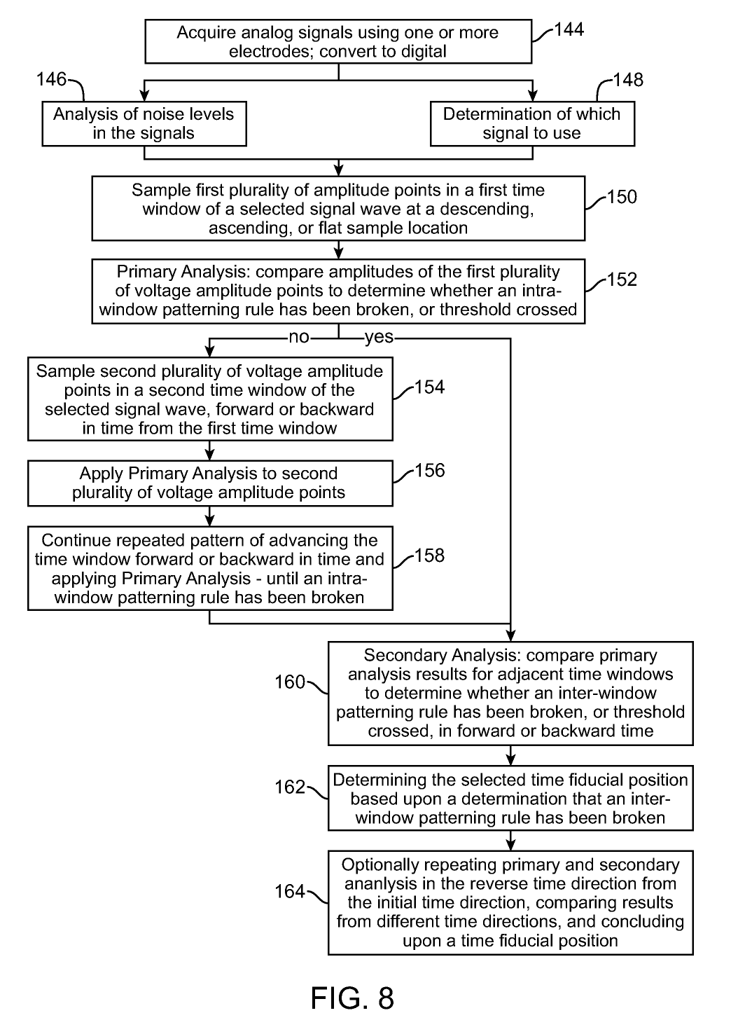

It is important to note that the primary and secondary analysis techniques described herein are broadly applicable. The previously discussed scenarios have involved, among other things, forward-in-time (i.e., in the direction as the events, such as ECG signals, occurred in real time) windowing analysis to determine the timing position of clinically relevant fiducials such as Tend associated with the end of a descending-in-amplitude signal wave such as a Twave. The subject primary and secondary analysis techniques may also be applied in reverse time as well as forward time, for ascending, descending, and flat signal waves to determine the positioning of various fiducial locations of interest on a given signal wave or set thereof. FIG. 7 illustrates a reverse-in-time, or “reverse time” embodiment, and FIG. 8 illustrates an embodiment generalized further for applicability beyond ECG signal waves—to any kind of signal wave.

Referring to FIG. 7, ECG signals may be acquired and converted (30), noise levels analyzed (32), and selected (34) in a similar manner as described in reference to FIG. 3. Subsequently, a first window sampling (130) may be conducted for a selected signal wave, and primary analysis conducted (132), followed by continued primary analysis of additional windows (134, 136, 138), looking at the signal wave data in reverse time and moving the sampling window successively backward in time along a signal trace (as plotted, for example, in amplitude versus time on Cartesian coordinates as in FIGS. 2A-2D—but proceeding to the left, or backwards in time, with the moving sampling window). Secondary analysis may be conducted for the reverse time scenario once an intra-window patterning rule has been broken to determine if an inter-window patterning rule has been broken (140), subsequent to which a selected fiducial timing position may be determined based upon the inter-window patterning rule breakage (142). In one embodiment, it may be desirable to only conduct forward-in-time analysis, as described in relation to FIGS. 2A-6. In another embodiment, it may be desirable to only conduct reverse-in-time analysis, as described herein in reference to FIG. 7. In another embodiment, it may be desirable to conduct both forward and reverse time analysis. These techniques may be utilized to determine the positions of all ECG fiducial locations, such as the timing locations of the start of the Pwave, the apex of the Pwave, the start of the P-R segment, the end of the P-R segment, the Q point, the R point, the S point, the beginning of the S-T segment, the end of the S-T segment, the apex of the Twave, the end of the Twave, starts, apices, and ends of any Uwaves, etcetera.

As discussed above, the inventive primary and secondary analysis may also be applied to other signal waves or traces, such as additional human electronic signal traces such as electroencephalogram (“EEG”) signals, electromyogram (“EMG”) signals, and the like, and other biological and nonbiological signal waves. A generalized embodiment is illustrated in FIG. 8. Referring to FIG. 8, analog signals may be acquired using one or more electrodes and converted to digital (144), after which noise levels may be analyzed (146) and targeted signals determined (148). Windowing primary analysis may be conducted for a first sampling window on the targeted signal wave, which may be at a location in the signal wave that is descending (as in the case of a descending Twave signal wave portion when considered in forward time), ascending, of flat in forward or reverse time (150, 152), and such analysis may be repeated with successive sampling window moves (154, 156, 158), until a primary patterning window has been broken, subsequent to which secondary analysis (160) may be conducted and a fiducial position determined (162). Optionally, an opportunity to also conduct similar analysis in the opposite-in-time windowing direction may also be exploited, and the results of the different directions compared and used for final fiducial position determination.

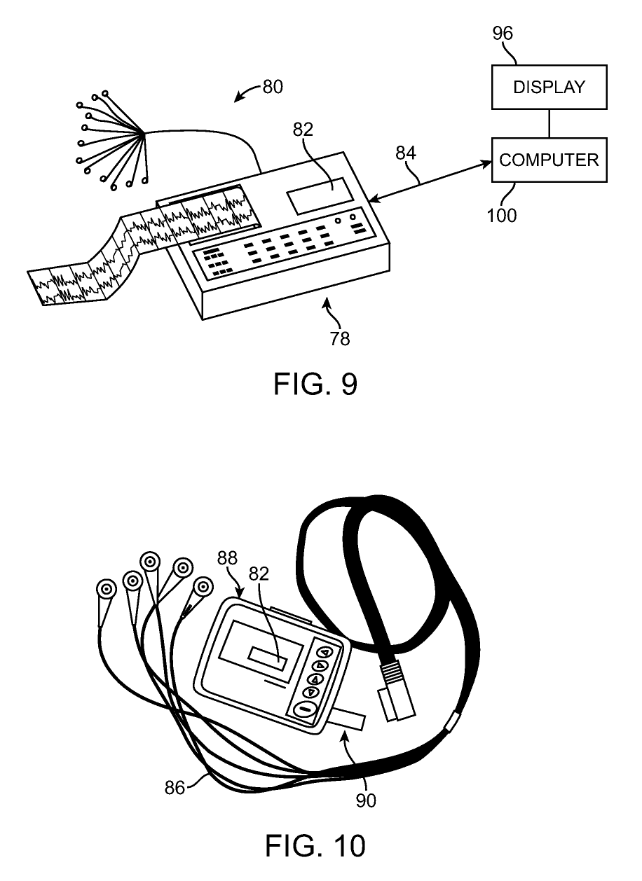

In practice, the techniques described in reference to FIGS. 2A-8 may be conducted on one or more computing systems, such as a personal computer, utilizing customized software, semi-customized software based, for example, on spreadsheets or customized configurations in applications such as the software package available under the tradename LabView® by National Instruments, Inc., and/or hardware configured to run embedded software. In some embodiments, it is preferred to have pertinent systems electronically integrated to facilitate realtime or near-realtime analysis in accordance with the techniques described above. For example, referring to FIG. 9, in one embodiment, an ECG acquisition system (78) and associated electrodes (80) preferably are integrated with a computer (100) using a wired or wireless coupling (84) whereby the computer (100) may receive and/or request data from the ECG system (78), and control activities and/or receive information from an embedded device (88), such as a card comprising integrated circuits and/or memory (and in one embodiment housed in a card housing and comprising an electromechanical card interface to connect with a bus comprising the ECG system), an application specific integrated circuit (“ASIC”), or a field programmable gate array (“FPGA”), each of which preferably would be configured to conduct primary and/or secondary analysis on raw data received by the ECG system (78) form the electrodes (80), in accordance with any instructions or control sequences that may be received from the computer (100), should the computer be connected at the time of sampling or before sampling. Referring to FIG. 10, an ambulatory, portable, Holter style ECG system (88) may also be similarly coupled to an embedded device (82) configured to conduct primary and/or secondary analysis based upon raw data received by such system (88) from an operably coupled electrode set (86). A bus or connector (90) may be provided for computing system (not shown) connectivity.

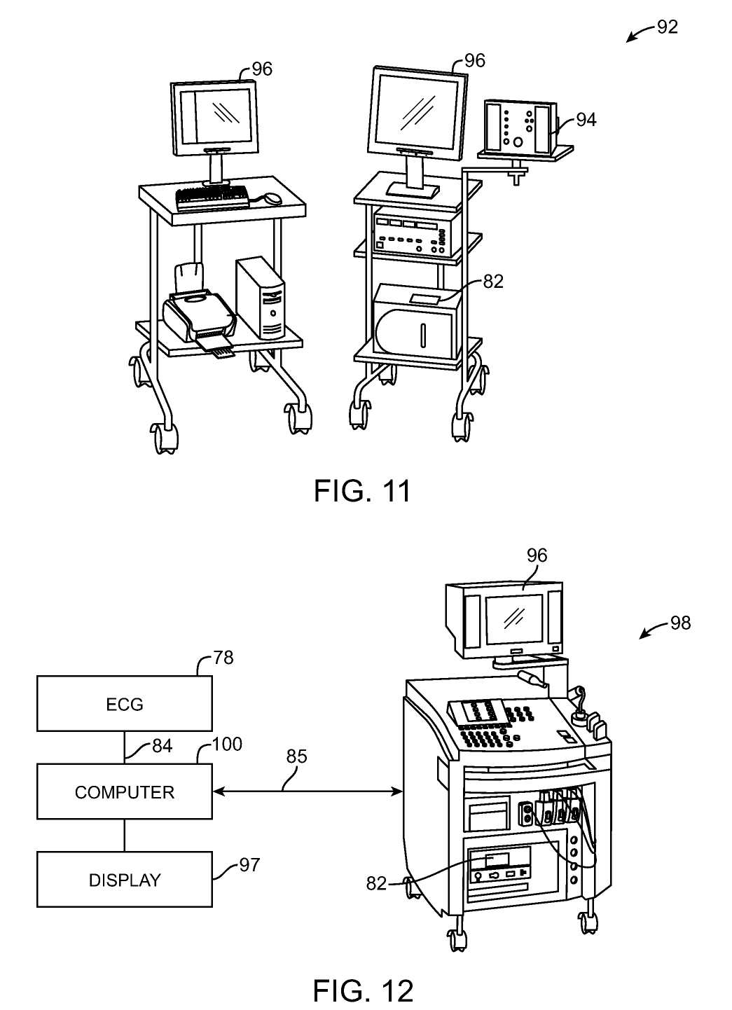

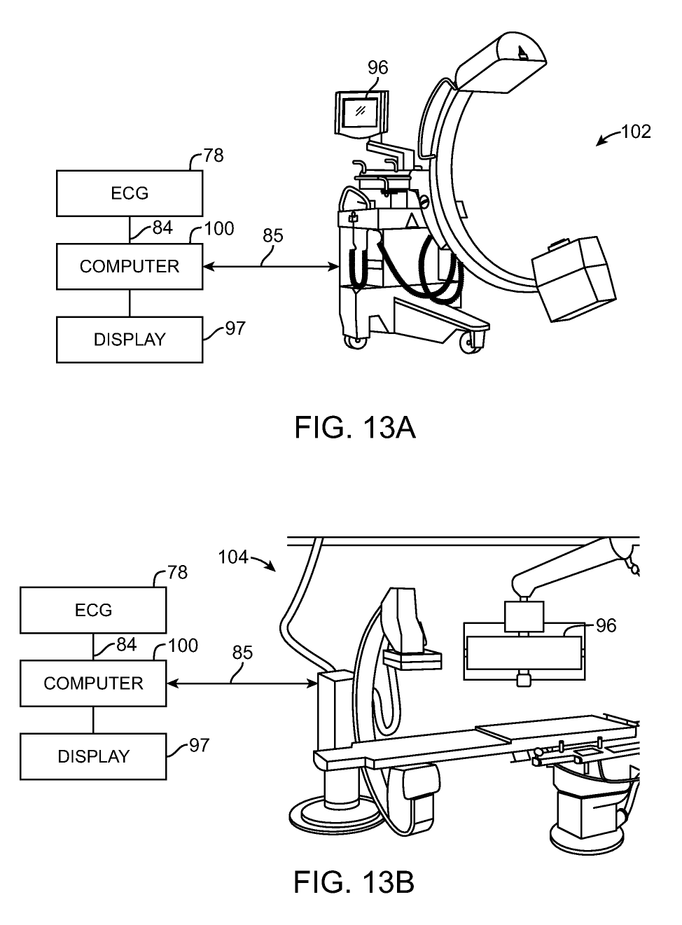

Referring to FIGS. 11-13B, other medical information processing systems commonly associated with ECG signal processing may also be desirably integrated with or embedded with primary and secondary processing infrastructure, in accordance with the present invention. For example, referring to FIG. 11, an electrophysiology mapping system (92), such as those available from Biosense Webster under the tradename CartoXP®, may also be operably coupled to an embedded device (82) configured to conduct primary and/or secondary analysis based upon raw data received by such system (92) from an operably coupled electrode set (not shown) coupled to an electrode connectivity bus panel (94). Tend and other results may be directed to the one or more displays (96). Referring to FIG. 12, an echocardiography system (98), such as those available from Siemens Medical Systems, Inc. under the tradename Sequoia®, may be operably coupled to a computing system (100) and an ECG system (78). An embedded device (82) configured to conduct primary and/or secondary analysis based upon raw data received from the ECG system (78), may be coupled to any one of the ECG system (78), as in FIG. 9, the computing system (100), or the echocardiography system (98). Data pertinent to the primary and secondary analysis preferably may be directed to either of the echocardiography display (96) or the computing system display (97). Similarly, referring to FIGS. 13A and 13B, a relatively simple fluoroscopy system (102), such as that depicted in FIG. 13A, or a more complex angiography system (104), such as that depicted in FIG. 13B, may be operably coupled and/or embedded with a device configured to conduct primary and/or secondary analysis based upon raw data received by electrodes operably coupled to a computing system (100), associated ECG system (78), the embedded device, or other system. Connectivity of the various components of such system configurations, such as the processor, memory device, and operating room electronic device, may be conducted using Ethernet, wireless technologies, and/or communication protocols such as TCPIP, FTP, or HTTP.

While multiple embodiments and variations of the many aspects of the invention have been disclosed and described herein, such disclosure is provided for purposes of illustration only. For example, wherein methods and steps described above indicate certain events occurring in certain order, those of ordinary skill in the art having the benefit of this disclosure would recognize that the ordering of certain steps may be modified and that such modifications are in accordance with the variations of this invention. Additionally, certain of the steps may be performed concurrently in a parallel process when possible, as well as performed sequentially. Accordingly, embodiments are intended to exemplify alternatives, modifications, and equivalents that may fall within the scope of the claims.

1. A system for determining a signal wave transition point, comprising:
a. a memory device configured to store data pertinent to one or more signal waves sampled from electrodes operably coupled to one or more tissue structures; and
b. a processor operably coupled to the memory device and configured to access the data and determine a transition point associated with the one or more signal waves, the processor configured to
1) sample a first plurality of points of a signal wave in a first time window, the first plurality comprising at least a first-in-time point and a last-in-time point within the first time window;
2) sample a second plurality of points of the signal wave in a second time window different in time from the first time window, the second plurality comprising at least a first-in-time point and a last-in-time point within the second time window;
3) compare the values of the first plurality relative to each other to determine whether an intra-window patterning rule has been broken; and
4) conduct a secondary analysis subsequent to determining that an intra-window patterning rule has been broken, the secondary analysis comprising comparing the values of the second plurality relative to each other to determine whether the intra-window patterning rule has been broken.

2. The system of claim 1, further comprising a display operatively coupled to the processor, wherein the processor is further configured to cause a graphical image of at least one of the one or more signal waves to be depicted on the display, including a graphical indication of a respective pertinent transition point on each of the one or more signal waves.

3. The system of claim 1, wherein the processor and memory device are operatively coupled to one or more printed circuit boards.

4. The system of claim 3, wherein the one or more printed circuit boards comprise a card having a common housing and an electronic interface bus.

5. The system of claim 3, wherein the card is configured to interface with a personal computer.

6. The system of claim 1, wherein the processor and memory device comprise an application specific integrated circuit configured to be embedded into a parent electronic device.

7. The system of claim 1, wherein the processor and memory device comprise a field programmable gate array configured to be embedded into a parent electronic device.

8. The system of claim 1, wherein the processor and memory device are operatively coupled to an operating room electronic device selected from the group consisting of an electrophysiology mapping system, an echocardiography system, and a fluoroscopic imaging system.

9. The system of claim 8, wherein the processor and memory device are operably coupled to the operating room electronic device by an Ethernet connection.

10. The system of claim 9, wherein at least two of the processor, memory device, and operating room electronic device are configured to communicate with each other via a protocol selected from the group consisting of TCPIP, FTP, and HTTP.

11. The system of claim 1, wherein the processor and memory device comprise a personal computer.

12. The system of claim 1, wherein the processor and memory device are operatively coupled to an analog signal acquisition system.

13. The system of claim 12, wherein the analog signal acquisition is operatively coupled to one or more electrodes.

14. The system of claim 12, wherein the analog signal acquisition system is selected from the group consisting of an electrocardiogram system, an electroencephalogram system, and an electromyogram system.

15. The system of claim 1, wherein the processor and memory device are enclosed within an implantable housing.

16. The system of claim 15, wherein the memory device is operatively coupled to an external computing system and configured to exchange data with the external computing system by wire, or wirelessly.

17. The system of claim 14, wherein the analog signal acquisition system comprises an ambulatory Holter monitor.

18. The system of claim 1, wherein the processor is configured to sample the second plurality of points in the second time window at least partially forward in time from the first time window.

19. The system of claim 1, wherein the processor is configured to sample the second plurality of points in the second time window at least partially backward in time from the first time window.

20. The system of claim 18, wherein the signal wave containing the second plurality of points being sampled is descending in amplitude versus time, and wherein a signal wave transition point is determined by the processor based upon an end of a descent of the descending signal wave.

21. The system of claim 18, wherein the signal wave containing the second plurality of points being sampled is ascending in amplitude versus time, and wherein a signal wave transition point is determined by the processor based upon an end of an ascent of the ascending signal wave.

22. The system of claim 19, wherein the signal wave containing the second plurality of points being sampled is descending in amplitude versus reverse time, and wherein a signal wave transition point is determined by the processor based upon an end of a descent of the descending signal wave in reverse time.

23. The system of claim 19, wherein the signal wave containing the second plurality of points being sampled is ascending in amplitude versus reverse time, and wherein a signal wave transition point is determined by the processor based upon an end of an ascent of the ascending signal wave in reverse time.
